(19) United States
(12) Patent Application Publication
(10) Pub. No.: US 2009/0124669 A1
(43) Pub. Date: May 14, 2009

(54) Structural Analogues of Avenanthramides, Their Use in Compositions Useful in the Treatment of Dermatological Disorders

(75) Inventor: Carlo Ghisalberti, Milano (IT)

Correspondence Address: OHLANDT, GREELEY, RUGGIERO & PERLE, LLP, ONE LANDMARK SQUARE, 10TH FLOOR, STAMFORD, CT, 06901, US

(73) Assignee: RELIVIA S.r.l

(21) Appl. No.: 11/884,717

(22) PCT Filed: Feb. 21, 2006
(86) PCT No.: PCT/EP2006/060133

§ 371 Date: Aug. 20, 2007

(30) Foreign Application Priority Data
Feb. 21, 2005 (IT) MI2005A000261

(51) Int. Cl. A61K 31/4415; A61K 31/4245; A61K 31/455; A61K 31/41; A61K 31/34; A61K 31/195; C07D 333/20; C07D 271/10; C07D 249/16; C07D 213/78; C07D 307/02

(52) U.S. Cl. 514/350; 514/357; 514/381; 514/563; 514/352; 514/438; 514/471; 514/364; 549/493; 549/74; 548/257; 548/144; 546/297; 546/310; 546/315; 562/450; 514/534

(57) ABSTRACT

Novel compounds for the prevention and treatment of dermatological disorders with an immunoallergic, hyperproliferative and inflammatory component, and a method of producing such compounds. The compounds are bioisosteres of substances with avenanthramide structures.

### STATE OF THE ART

The present invention relates to bioisosteres, aza- and thio-analogue derivatives of substances with avenanthramide structure useful for preparing pharmaceutical compositions effective in the treatment of dermatological diseases of immunoallergic, hyperproliferative and inflammatory type.

Eczemas and psoriatic forms account for approximately 30-50% of all dermatological problems and comprise a range of dermatitis of composite immunological, hyperproliferative and inflammatory etiologies. A characteristic of the disease is its cyclical course, characterised by a multiple stages symptomology. The reasons for the onset of single eczematous reactions are extremely heterogeneous and in some cases their complexity has not been clarified. The classification is therefore based mainly on the origin of the triggering causes, on the nature and significance of the immunological component and on the basic predisposition.

The most important forms are psoriasis, contact eczema, seborrheic dermatitis and (constitutional) atopic dermatitis. Typical of the acute stage is an aggressive, exudative inflammation with erythema, edema, blisters, erosions and secretions, followed by desquamations and scabs. The subacute stage, immediately after, still presents exudative symptoms, but also specific signs of chronic inflammation, such as vesicular papules. In the third stage, known as the chronic stage, the inflammatory processes decrease and a thickening of the skin surface (lichenification) and stratum corneum (hyperkeratosis) takes place with consequent appearance of chapping. A relapse of the chronic form causes instead the simultaneous development of both lichenification and secretory phenomena. A subjective cutaneous symptom present in all stages is an intense itching characterised by stages of relapse. Due to continuous scratching, abrasions form which become encrusted in blood causing further itching attacks, thus providing at the same time a favourable medium for the appearance of fungal, viral or bacterial infections.

Substances with avenanthramide structure are found to be among the many studied for the treatment of erythematous dermatological diseases. U.S. Pat. No. 3,490,422 and JP487359 claim in particular the anti-allergic action and use of 2-{[3-(3,4-dimethoxyphenyl)acryloyl]amino}benzoic acid, or tranilast, in atopic dermatitis.

Tranilast has had an important clinical history, becoming a multi-functional drug i.e. able to perform pharmaceutical functions in various therapeutic contexts. Indeed, tranilast seems not only to act as an anti-inflammatory but also as a fibrosis and proliferation inhibitor, for example of vascular smooth muscle, in addition to exerting a stabilizing and suppressing effect on mastocytomas, see Katoh N et al. (J. Dermatol. (Tokyo) 23, 335-9, 1996). Researchers at Kyoto and Shinshu universities have verified the dose dependent ability of tranilast to inhibit the growth of uterine leiomyomas with no cytotoxic effect. Tranilast seems able to suppress CDK2 activity via induction of p21 (waf1) and p53 (Shime H et al., J Clin Endocrinol Metab 2002; 87:5610-5617).

Tranilast belongs structurally to the avenanthramide family, compounds present in oat glumes, they being anthranilic acid amides and substituted cinnamic acids identified in fractions of hydroalcohol extracts of oat. The main ones are known as A, B and C avenanthramides-5-hydroxy-2-{[3-(4-hydroxyphenyl)acryloyl]amino}benzoic acid, 5-hydroxy-2-{[3-(4-hydroxyphenyl-3-methoxyphenyl)acryloyl]amino}benzoic acid and 5-hydroxy-2-{[3-(3,4-dihydroxyphenyl)acryloyl]amino}benzoic acid respectively—in addition to other analogues present in oats at lower concentrations as identified by Collins W (J. Agric. Food Chem. 37, 60-66, 1989).

Vollhardt and co-researchers have again evidenced the lenitive and antioxidative behaviour of avenanthramides, proposing them as ingredients for cosmetic use (Proceedings 21st IFSCC Internat Congress, 395; Berlin, 2000).

DE10254872 (WO2004/047833) refers to the property of avenanthramides to inhibit histamine release, confirming their potential as ingredients of anti-inflammatory and/or lenitive topical preparations.

### SUMMARY OF THE INVENTION

The present invention relates to bioisosteres of substances with avenanthramide structure for the prevention and treatment of dermatological disorders with an immunoallergic, hyperproliferative and inflammatory component. The active principles of the present invention are compounds of formula (I):

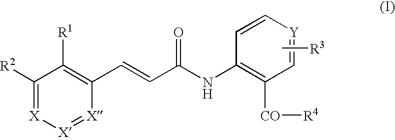

where:
X, X′, X″ and Y each independently represents N,N→O or C—R⁵;
or the X′=X″ group represents a sulfur atom S or an oxygen atom O, with X remaining as aforedefined;
R⁴ represents —OH, O-benzyl, O-phenyl, —CH₂—CH(NHR⁶)—COOR⁸
or the —CO—R⁴ group represents a bioisostere of the —COOH group;
R¹, R², R³ and R⁵ each independently represents —H, OH CO—R⁴, —NH₂, —NH—CO—R⁴, —O—COR⁷, -Halogen, —OR⁷, —R⁷;
R⁶ represents H or —COR⁷;
R⁷ and R⁸ each independently represents a linear or branched saturated or unsaturated C₁₋₂₀ alkyl group, optionally substituted: preferably a C₁₋₃ alkyl, even more preferably a methyl group;
with the proviso that at least one of X, X′, X″ and Y is different from C—R⁵ or if X, X′, X″ and Y are all equal to C—R⁵, then at least one of R¹, R² and R⁵ is different from —H, —OH and —OR⁷;
or a salt, solvate or prodrug thereof.

Also described is a procedure for the synthesis of compounds of formula (I) by condensation of compounds of formula (a) and (b).

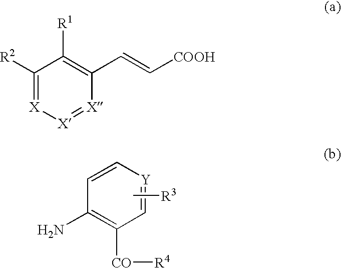

where R¹, R², X, X′, X″, Y, R³, R⁴ have the previously indicated meanings.

The present invention also includes: pharmaceutical compositions containing the compounds of formula (I) and their use in the treatment of the aforesaid dermatological disorders; cosmetic compositions containing the compounds of formula (I), and their use in the cosmetic treatment of skin.

### DETAILED DESCRIPTION OF THE INVENTION

The term “bioisostere of the —COOH group” defines a biological equivalent of the carboxyl group, e.g. as described by Greenwood J R et al. “Heterocycles as bioisosteres for the ω-carboxylate moiety of glutamate in AMPA receptor agonists: A review and theoretical study” in Internet Journal of Chemistry 1, 38 (1998). Illustrative examples of bioisosteres of the —COOH group for the substance of formula (I) are:

a hetero-pentatomic ring selected in the following group:

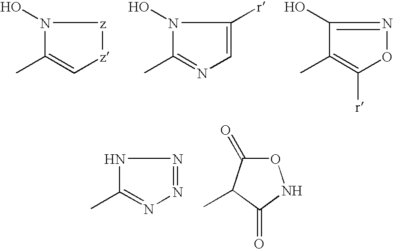

where z and z′ represent N or C-r′; r′ represents H or C₁₋₃ alkyl, preferably H or methyl;

a group containing a hetero-atom with a double bond of electron-attracting character included in the following group:

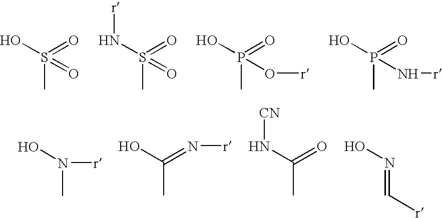

where r′ represents H or C₁₋₃-alkyl, preferably H or methyl.

The term “possibly substituted” refers to the substitution of a hydrogen with a monovalent or divalent radical. Suitable substituent groups include, for example, hydroxyl, nitro, amino, imino, cyano, halo, thio, thioamido, amidino, oxo, oxamidino, methoxamidino, imidino, guanidino, sulfonamido, carboxyl, formyl, C₁₋₆-alkyl, haloC₁₋₆-alkyl, C₁₋₆-alkoxy, haloC₁₋₆-alkoxy, C₁₋₆-alkoxyalkyl, alkylcarbonyl, arylcarbonyl, aralkylcarbonyl, heteroarylcarbonyl, heteroaralkylcarbonyl, alkylthio, aminoalkyl, cyanoalkyl and the like. The substituent group can itself be substituted. The substituents within the substituent groups can be, for example, carboxyl, halo, nitro, amino, cyano, hydroxyl, C₁₋₆-alkyl, C₁₋₆-alkoxy, amino-CO—, —SR, thioamido, —SO₂R or cycloalkyl, where R is typically hydrogen, hydroxyl or C₁₋₆-alkyl. When the secondary substituent comprises a linear chain group, the substitution can occur either within the chain (e.g. 2-hydroxypropyl, 2-aminobutyl, etc.) or at the chain terminus (e.g. 2-hydroxyethyl, 3-cyanopropyl, etc.). Secondary substituents can have linear, branched or cyclic carbon or heteroatom chain configurations.

These and other terms can be easily understood from the international patent literature, for example U.S. Pat. No. 6,727,273, with explanatory examples.

The solvates are forms of hydration, solvation and hydrolipid complexation, such as liposomes, nanospheres, microemulsions etc., and inclusion complexes, e.g. produced by incorporation into cyclodextrins and other cross-linked polymers such as PVP XL, dextrans, fullerenes.

The salts of the substances of formula (I) are formed with physiologically acceptable acidic or basic counterions. Inorganic base salts include sodium, potassium, lithium, ammonium, calcium, magnesium, zinc etc. Organic base salts include amines, e.g. isopropylamine, diethanolamine, triethanolamine, tripropylamine, ethanolamine, 2-dimethylaminoethanol, tromethamine, dicyclohexamine, lysine, arginine, histidine, caffeine, procaine, hydrabamine, choline, betaine, ethylenediamine, glucosamine, methylglucamine, theobromine, piperazine, piperidine, etc.

Acid addition salts are formed with inorganic acids such as hydrohalic, sulphuric, nitric, phosphoric acids etc., or with organic acids such as acetic, glycolic, pyruvic, oxalic, malic, malonic, succinic, maleic, fumaric, tartaric, citric, benzoic, cinnamic, mandelic, methane- or p-toluene-sulfonic, salicylic, azelaic, undecylenic, etc.

Prodrugs insertable into other functional groups possibly present in the structure of the substances of formula (I), are groups releasable in vivo, for example as illustrated by Sloan in “Prodrugs: Topical and ocular drug delivery” NY Marcel Dekker; Larsen & Ostergaard in “Textbook of drug design and discovery”, Chapter 14, London-NY, Tailor & Francis.

A preferred subgroup of compounds of formula (I) is one in which Y is chosen from N and N→O.

A preferred second subgroup is one in which at least one from X, X′, X″ is chosen from N and N→O. A third preferred subgroup is one in which the X′=X″ group represents S or O.

For the purposes of the present invention substances of structures (II)-(XVI) are preferred:

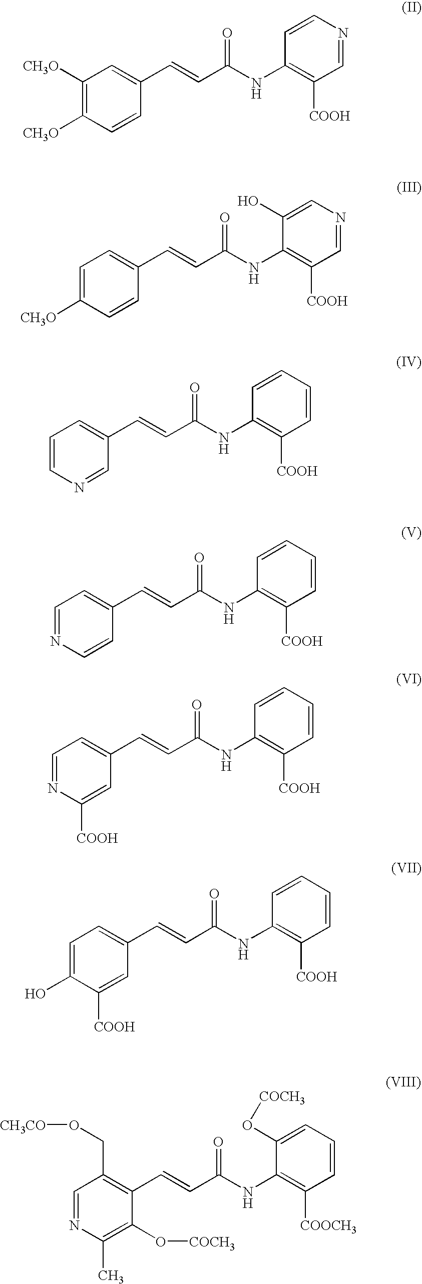

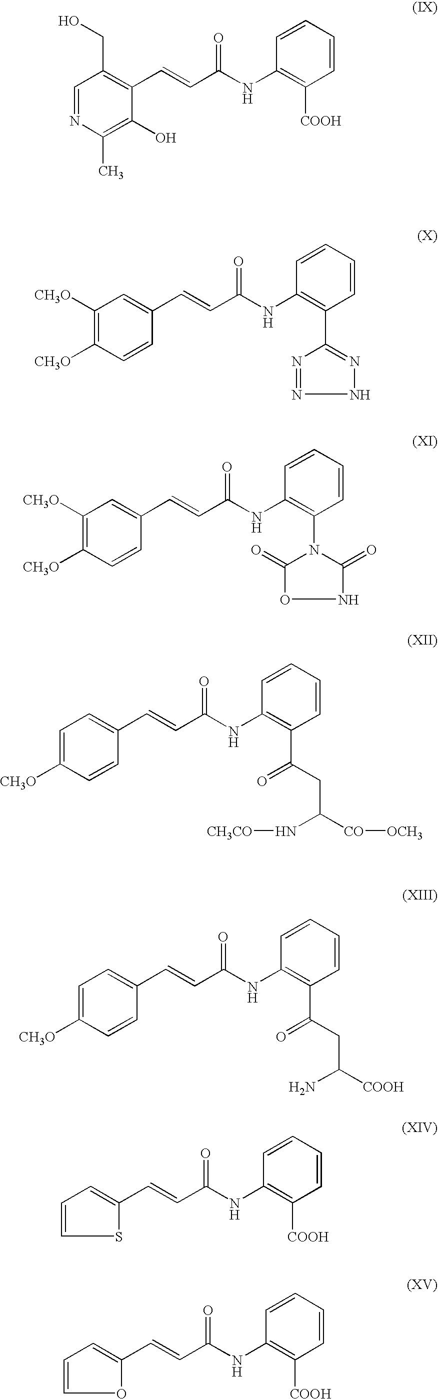

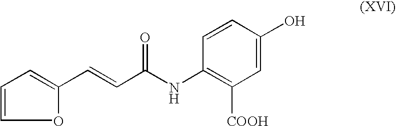

The synthesis strategies for the substances of formula (I) preferably comprise condensation of an (aza)cinnamic-type compound of formula (a) with an (aza)anilinic-type compound of formula (b):

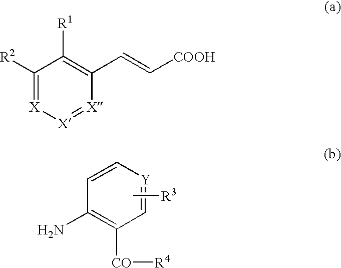

wherein R¹, R², X, X′, X″, Y, R³, R⁴ have the previously indicated meanings.

The condensation step may be carried out by the reaction of the optionally protected compound (b) with the acid halide or anhydride of the optionally protected compound (a), previously prepared by contacting the carboxy group with an inorganic acid halide, such as SOCl₂, PCl₆, PBr₃ or PCl₅, or alternatively, with oxalyl chloride, typically in the presence of an amine as acid-scavenger, e.g triethylamine, diisopropylethylamine, N-methylmorpholine and the like.

Alternatively, compounds (a) and (b) can be condensed using any conventional coupling reagent including carbodiimides such as dicyclohexylcarbodiimide, N,N′-carbonyldiimidazole, N-hydroxysuccinimide, 1-hydroxybenzotriazole, etc. to facilitate the coupling of carboxylic moiety with the amine group.

Functional groups, such as amino, hydroxyl, or carboxyl groups optionally present in the compounds (a) and/or (b) need to be protected before any reaction is initiated, wherein the removal of the protecting group may be the final step in a particular reaction. Suitable protecting groups for such functionality are apparent to those skilled in the art. For specific details see “Protective Groups in Organic Synthesis”, Wiley Interscience, TW Greene, PGM Wuts.

Therefore, compounds (a) and (b) containing —OH, —NH₂ or further —COOH groups will be protected prior to the condensation, e.g. O-protected as O—Ac, COO-protected as methyl esters, N- or O-protected by t-butyloxycarbonyl, t-amyloxycarbonyl or benzyloxycarbonyl, and so on.

It is not always necessary to isolate and purify these protected derivatives. For example, an O-protected intermediate (a) can be reacted with chloroformate or SOCl₂ and the resulting alkyl carbonate mixed anhydride or acid chloride may be used without isolation and purification in the condensation step.

Conversely, a deprotection reaction may be necessary in case the free —OH, —NH₂ or —COOH were found to be more biologically active than their protected forms.

The present compounds have a distinct anti-proliferative, immuno-suppressive and anti-inflammatory activity: due to these properties, they have proved useful in the treatment and/or prevention of dermatological disorders, in particular those with an immunoallergic, hyperproliferative and inflammatory component. Examples of such disorders are eczemas (e.g. contact eczema, seborrheic eczema), psoriases, dermatitis (e.g. atopic; due to drugs, contact with an irritant, contact with an allergen; seborrheic; due to diapers; infantile papular acrodermatitis), dermatological diseases of erythematous character, fibrosis, lichen ruber planus, pityriasis rosea, autoimmune bullous diseases, urticaria and angioedema.

An aspect of the present invention is therefore the use of one or more of the aforesaid compounds of formula (I) for preparing a drug useful in the treatment and/or prevention of said dermatological disorders.

A further aspect of the invention are pharmaceutical compositions comprising one or more compounds of formula (I) in a therapeutically effective quantity and dermatologically/physiologically acceptable excipients and ingredients.

The dermatologically/physiologically acceptable excipients and ingredients can also include a physiologically acceptable vehicle that acts as diluent, dispersant or carrier of the substance of interest in the composition. Vehicles other than water can include liquid or solid emollients, silicones and solvents. Oils and lipids can be combined with water by means of emulsifiers and surfactants; moreover preservatives, pigments, opacifiers, fragrances and other cosmetic ingredients from the INCI list can be included.

In a preferred embodiment, the compositions of the invention are utilized in combination with other antioxidant substances, e.g. those usable for such purposes in the cosmetic field.

These non-limitatively include: retinoids (e.g. retinoic acid, retinol), carotenoids (e.g. α- and β-carotene), xanthophylls (e.g. lutein, zeaxanthin), tocopherols (e.g. α- and γ-tocopherol) and derivatives, salicylates, ascorbates, alpha-hydroxy acids (e.g. glycolic acid, lactic acid, citric acid, their salts and esters), plant flavonoids and polyphenols (rutin, hesperidin, quercetin, catechin, gallic acid and gallates, OPC, tannic acid), melatonin, melanin, carnosine and urocanic acid, thiol amino acids (cysteine, cystine, methionine, their amides and salts), lipoates, etc.

Furthermore, in dermoprotective applications, the substances of the present invention can be utilized in combination with other substances of lenitive character such as alpha-bisabolol, azulene, guaiazulene, 18-beta glycyrrhetic acid.

The substances of formula (I) are also suitable for combination with other active principles.

Illustrative examples are polysaccharides with wound healing and lenitive and/or angiogenic action comprising high MW hyaluronan (HA) and low MW hyaluronan (OHA); sulfated glycosaminoglycans such as heparin, heparan, chondroitin and keratan sulfate; polygalactomannans such as guar gum, poly-agaroses and poly-agaropectins such as agar-agar; poly-uronates such as algins and alginates; polygalacturonates alone and in combination with arabinose and galactose such as pectin and pectinates; mixed polysaccharides such as acacia gum (arabic), karaya gum, tragacanth gum (bassorin), K-carrageenan and λ-carrageenates; polymanno-gluco-glucuronates such as xanthan gum, acetylated polymannoses (aloe gel), chitin and chitosan; dextranomer; etc.

Particularly preferred for the present invention are polysaccharides of Dglucuronic acid and N-acetyl-glucosamine (GlcA/GlcNAc), known as hyaluronan, either of high molecular weight (HA) with wound healing activity or low molecular weight oligosaccharides (OHA) with angiogenic action useful for restoring possible depletion of the vascular system in skin or the mucosa. Additional preferred polysaccharides for wound healing and lenitive action are acetylated polymannoses (acemannans) of aloe, and alginates.

Other illustrative examples are polyunsaturated fatty acids which include C₁₆-C₂₄ unsaturated fats with at least two double bonds of the omega-3 and -6 series, such as gamma-linoleic, alpha-linoleic, linolenic, homo-gamma-linolenic, columbinic, eicosa-(n-6,9,13)-trienoic, timnodonic, arachidonic, docosapentaenoic, eicosahexaenoic, etc.

For the purposes of the invention the polyunsaturated fatty acid can be present in free form or as a triglyceride or other ester. Most frequently vegetable oils with high polyunsaturates content are used, such as borage oil, flax seed oil, soybean oil, deodorized fish oil, algal oil (species selected for high DHA content), avocado oil, walnut oil, hemp oil, rose oil, almond oil, corn oil, grape seed oil, safflower oil, sesame oil, sunflower oil, wheatgerm oil, jojoba oil, olive oil, etc.

Further examples are steroidal (cortisonic) and non-steroidal anti-inflammatories.

These latter include the following classes of substances:

1) oxicams, such as piroxicam, isoxicam, tenoxicam and sudoxicam;
2) salicylates, such as aspirin, disalcid, benorylate, briflunisal, safaprin, solprin and diflunisal;
3) acetic derivatives such as diclofenac, indomethacin, sulindac, tolmetin, fentiazac;
4) fenamates, such as mefenamic, flufenamic, niflumic and tolfenamic acids;
5) propionic derivatives, such as ibuprofen, naproxen, benoxaprofen, flurbiprofen, ketoprofen, fenoprofen, oxaprozin, suprofen, alminoprofen, and tiaprofenic acid;
6) pyrazoles, such as phenbutazone, oxyphenbutazone, feprazone, azapropazone and trimethazone.

Other examples are: topical antimycotics (e.g. imidazolic and triazolic derivatives such as miconazole, econazole); emollients and barrier creams (e.g. ZnO pastes); cicatrizants (e.g. acetylcysteine, proteolytic enzymes); topical antibiotics (e.g. fusidic acid, gentamicin, mupirocin); topical chemotherapeutics (e.g. sulfonamides, silver sulfadiazine); antivirals (e.g. aciclovir); antiseptics and disinfectants (e.g. biguanidines and amidines such as chlorhexidine+cetrimide, borates, povidone-iodine, iodine, quaternary ammonium compounds such as benzalkonium chloride, mercurials such as merbromin, silver, H₂O₂, NaOCl); anti-acne agents (e.g. retinoic acid, meclocycline).

Treatment with the substances of formula (I) of the invention can also be combined with systemic antibiotic therapy with: tetracycline (e.g. doxycycline), amphenicols (e.g. chloramphenicol); beta-lactams (wide spectrum penicillins; lactamase-resistant penicillins; monobactams such as azthreonam; carbapenems such as meropenem, imipenem-cilastatin); sulfamides (e.g. sulfadiazine, sulfamethoxazole-trimethoprim); macrolides and the like (e.g. erythromycin, clarithroymycin, azithromycin, lincosamides, clindamycin, streptogramins); aminoglycosides (e.g. streptomycins, gentamicin, amikacin, netilmicin); quinolones (e.g. ciprofloxacin, norfloxacin, lomefloxacin, levofloxacin, moxifloxacin); glycopeptides (e.g. vancomycin, teicoplanin, polymyxin, colistin); imidazoles (e.g. metronidazole); nitrofurans (e.g. nitrofurantoin); and other antibacterials (e.g. linezolid).

Said substances can be administrated by various routes: oral, intravenous, intraperitoneal, intragastric infusion, enema, infusion via portal vein, inhalation, etc.

The compositions of the present invention contain the compound of formula (I) in a quantity between 0.01 and 80% by weight of the composition, preferably between 1 and 20% by weight.

The dosage of compound (I) is generally between 0.01 and 2000 mg/day.

Such compositions can be formulated in a lipophilic base (or, if required, in an aqueous vehicle) for topical application as dermatological formulations. A typical composition for topical use would contain between about 1 μg and 50 mg of active principle per gram of composition. The compositions of the present invention can also be formulated in the form of lotions, fluid creams, creams or gels. The compositions can be packaged in a suitable container based on the viscosity and application. For example, a lotion or fluid cream can be packaged in a bottle or a roll-on applicator, while a thick cream can be packaged in a small tube, pot or jar.

The compositions of the invention can be administered systemically by i.v., s.c. injection etc. of a previously sterilized solution, suspension or emulsion containing the substance of formula (I) using known techniques.

The compositions containing the substances of formula (I) can also be administered orally, sublingually, trans-rectally, by pulmonary inhalation, by techniques developed for release of drugs to avoid the gastro-hepatic route, and/or by injection method.

Clear results can be obtained for degenerative pathologies requiring the active principle to be supplied in a quantity sufficient to produce the required effects. The present therapeutic treatment is clearly intended to be combined, either simultaneously or alternately, with other treatments and topical or sistemic drugs currently prescribed for the aforedescribed disorders.

The compounds of formula (I) also provide lenitive, antioxidant and dermoprotective activities: these properties also render them useful in other than the pharmaceutical field, for solely cosmetic application. The present invention therefore extends to a method of cosmetic treatment for the skin characterised by the topical administration of a cosmetically effective quantity of one or more compounds of formula (I), in combination with excipients for cosmetic use. The invention also comprises the relative cosmetic compositions characterised by including one or more compounds of formula (I), combined with excipients for cosmetic use. The following examples serve to further illustrate the invention and should not be considered in any way limiting.

### EXAMPLES

### Synthesis Methods

### Condensation of (a) and (b)

PCl₃ (1.6 g, 2.3 eq) is added dropwise to a solution of a compound of formula (a) (1 eq) for example 3,4-dimethoxy-(aza)cinnamic and a compound of formula (b) (1.3 eq) for example (aza)methylanthranilate in anhydrous THF (30 ml), under nitrogen atmosphere. The solution is then heated at reflux for 5 hours. At the end of the reaction (TLC) the solvent is evaporated. The crude product is purified by flash chromatography on silica gel (e.g. hexane-ethyl acetate 9:1 v/v). The substance of formula (I) is obtained.

### Esterification of —OH to —O—Ac

Protection of the hydroxyl groups, particularly phenolics, by means of the acetate group is achieved with acetic anhydride in pyridine at ambient temperature in accordance with conventional methods.

### Hydrolysis of O—Ac Groups to —OH

A solution of a substance of formula (I) containing acetic esters of phenolic or hydroxyalkyl groups (0.26 mmoles) in MeOH/H₂O/25% NH₄OH 50:35:15 (10 ml) is heated to 50° C. for 1 hour. The solvent is removed under reduced pressure and the crude product purified by flash chromatography (eluent hexane/AcOEt 1:1) to obtain the corresponding hydrolysed product.

### Esterification of the —COOH Groups to —CO-OMe

Concentrated H₂SO₄ (4.3 ml) is slowly added dropwise to a solution of an intermediate of formula (a) or (b) containing —COOH groups (13.7 mmoles) in anhydrous MeOH (17.1) under nitrogen atmosphere. The reaction is left under agitation at reflux for 4 hours then poured into H₂O (10 ml) and basified by addition of NaHCO₃ to pH 9. The precipitate is filtered off through a buchner funnel and washed with MeOH. The corresponding esterified product is obtained.

### Hydrolysis of —CO-OMe Groups

A solution of a substance of formula (I) containing carboxylic groups in the form of methylester (0.17 mmoles) in 0.25 M NaOH in MeOH (13 ml) is heated at reflux for 14 hours followed by solvent removal under reduced pressure. The residue is redissolved in H₂O (5 ml) and acidified with concentrated HCl to pH 4. The precipitate is filtered off through a buchner funnel and washed with H₂O to obtain the corresponding hydrolysed product.

Protection of —OH or —NH₂ Groups by t-BOC

Anhydrous Et₃N (425 μl, 3 mmoles) and BOC₂O (1 g, 4.6 mmoles) are added to a solution of a substance of formula (b) (3 mmoles) in anhydrous CH₂Cl₂ (30 ml) under nitrogen atmosphere. The reaction is left under agitation for 12 hours, the solvent is removed under reduced pressure.

A variant of the preceding method is the following: substance of formula (b) 0.61 mmoles in CH₂Cl₂ (1 ml) and BOC₂O (440.4 mg, 2.02 mmoles) in CH₂Cl₂ (1 ml) are added to a suspension of NaOH (67.2 mg, 1.68 mmoles) and Bu₄NHSO₄ (5 mg, 0.012 mmoles) in CH₂Cl₂ (1 ml) at ° C. The reaction mixture is kept under stirring at ambient temperature for 12 hours then H₂O (10 ml) is added and extraction with CH₂Cl₂ is undertaken. The organic phase is dried over Na₂SO₄, filtered off and the solvent removed under reduced pressure.

The crude product obtained is purified by flash chromatography on silica, e.g. with hexane/ethyl acetate 75:25 as eluent.

Simultaneous hydrolysis of —O—Ac, —CO-OMe and —O-tBOC Groups

The methyl ester can also be hydrolysed directly with 0.25M NaOH in MeOH at reflux for 14 hours, with simultaneous removal of all protecting Groups. The subsequent work-up is carried out under classical conditions to give a substance of formula (I).

### Synthesis of Substance of Formula (X)

A classical method for obtaining the substance of formula (X) contemplates the conversion of N-(3′,4′-dimethoxy cinnamoyl) anthranilic acid or the relative nitrile, typically by reacting a hydrazoic acid source (e.g. sodium azide and ammonium chloride) with an acceptor such as the nitrile group in an inert solvent at high temperatures.

As an alternative, the Wittenberger and Donner synthesis is used using nitrites and trimethylsilyl azide (TMS-N₃) with catalysis by dialkyltin oxides in toluene at reflux.

### Synthesis of the Substance of Formula (XI)

The substance of formula (XI) can be obtained from N-(3′,4′-dimethoxy cinnamoyl) anthranilic acid as described by Weinstock et al. in J. Org. Chem., 1967, 32, 2823.

### BIOLOGICAL EXAMPLES

### T Cell Suppressive Activity

The selective apoptotic activity on murine thymocytes and Th1 cells can be evaluated by in vitro method according to Puccetti P et. All., Cell Death Different. (2002) 9:1069-1077.

Thymocytes from 4±6-week-old DBA/2 mice (Charles River, Calco, Italy) were enriched by passage through nylon wool columns. C57BU6-lpr/lpr mice (Jackson Laboratory, Bar Harbor, Me., USA) were used as such. Cell viability (>95%) as well as their characterisation as macrophages as main population (>99%) was confirmed by microscopy analysis and staining for nonspecific esterase.

### Material and Method

Cells were incubated with 10 mM of compounds of formula (I) for 24 h in RPMI medium containing 10% FCS. Afterwards, aoptosis was then measured by flow cytometry as described by Puccetti P et all., (1997) J. Immunol. 158:3593-3602. Briefly, cells were centrifuged after culturing and the pellets were gently resuspended in 0.3 ml hypotonic propidium iodide (PI) solution (PI, 50 mg/ml in 0.1% sodium citrate plus 0.10% Triton X-100; Sigma Chemical Co.). The tubes were kept at 48° C. in the dark for 1 h. The PI-fluorescence of individual nuclei was measured by flow cytometry with standard FACScan equipment (Becton Dickinson, Mountain View, CA, USA) with a incident light beam of a 488-nm argon laser and a 560-nm dichroid mirror (DM 570) and a 600-nm band pass filter (band width 35 nm) to collect the red fluorescence by PI DNA staining. The data were recorded in log scale in a Hewlett Packard (HP 9000, model 310; Palo Alto, Calif., USA) computer, where the % of apoptotic nuclei (subdiploid DNA peak in the DNA fluorescence histogram) was calculated with FACScan research software (Lysis II).

### Results

T cell apoptosis observed at concentrations around 10 mM of the substances of formula (I) ranges from 20% to 75%. These data indicate that the deletion of T lymphocytes may represent a major contribution of the substances of formula (I) to ameliorate dermatologic disorders sustained by immune, allergic, hyper-proliferative and inflammatory local reactions.

### COMPOSITION EXAMPLE

### Example 1

### Cream

100 g of emulsion contains:

| | | |
|---|---|---|
| Compound of formula (II) | 0.5 | g |
| Stearic acid | 1.75 | g |
| Propylene glycol monostearate | 2.7 | g |
| Bentone gel of caprylic/capric | 6.0 | g |
| propylene glycol | | |
| Isopropyl palmitate | 6.5 | g |
| Silicone fluid 345 | 3.0 | g |
| Sorbitan stearate | 1.8 | g |
| PEO-sorbitan stearate | 1.5 | g |
| Cetyl alcohol | 0.6 | g |
| UVA and UVB filters | 2.0 | g |
| Sodium EDTA | 0.1 | g |
| Aluminum silicate | 0.8 | g |
| Carboxymethylcellulose | 0.15 | g |
| Propylene glycol | 4.0 | g |
| Demineralized water | q.b. to 100 | g |

### Example 2

### High Internal Phase W/O Emulsion

100 g of emulsion contains:

| | | |
|---|---|---|
| Compound of formula (IV) | 0.3 | g |
| Retinol | 0.5 | g |
| Hydrogenated coconut oil | 5.9 | g |
| Oleyl-(2)-POE | 5.0 | g |
| Bentone 38 | 0.5 | g |
| MgSO₄ × 7H₂O | 0.3 | g |
| Demineralized water | q.b. to 100 | g |

### Example 3

### Anti-Erythema Preparation

100 g of emulsion contains:

| | | |
|---|---|---|
| Compound of formula (VIII) | 0.3 | g |
| Cyclomethicone | 2.0 | g |
| Cetearyl alcohol + Hydrogenated castor | 4.5 | g |
| oil PEG-40 + Na cetearyl sulfate | | |
| Octyl stearate | 3.0 | g |
| Castor oil | 4.0 | g |
| Glycerin | 3.0 | g |
| Carbopol | 0.3 | g |
| Hydroxypropylmethylcellulose | 0.3 | g |
| Octyl methoxycinnamate | 5.0 | g |
| Butyl-methoxy-dibenzoyl methane | 0.5 | g |
| Sodium EDTA | 1.5 | g |
| Demineralized water | q.b. to 100 | g |

### Example 4

### Anti-Dermatitis Cream

100 g of emulsion contains:

| | | |
|---|---|---|
| Compound of formula (XVI) | 0.2 | g |
| Beeswax | 1.5 | g |
| Almond oil | 13.0 | g |
| Xanthan gum | 0.5 | g |
| Superoxide dismutase (SOD) | 0.004 | g |
| Cyclopentadimethylsiloxane | 5.0 | g |
| Sucrose mono- and di-palmitate/- | 3.0 | g |
| stearate | | |
| Methylglucose sesquistearate | 3.0 | g |
| Stearic acid | 1.0 | g |
| Cetyl alcohol | 3.0 | g |
| Preservative | 0.3 | g |
| Demineralized water | q.b. to 100 | g |

### Example 5

### Anti-Eczema Cream

100 g of cream contains:

| | | |
|---|---|---|
| Compound of formula (III) | 0.3 | g |
| Fluid petrolatum | 2.0 | g |
| Cetyl alcohol-(10)-POE | 4.0 | g |
| Cetearyl alcohol | 4.0 | g |
| Triethanolamine | 1.75 | g |
| Butan-1,3-diol | 3.0 | g |
| Xanthan gum | 0.3 | g |
| Demineralized water | q.b. to 100 | g |

### Example 6

### Anti-Rosacea Hydroalcoholic Lotion

100 g of lotion contains:

| | | |
|---|---|---|
| Compound of formula (VI) | 1.5 | g |
| Tocopheryl acetate | 0.15 | g |
| Azelaic acid | 2.0 | g |
| Ethanol 95° | 20 | g |
| Demineralized water | q.b. to 100 | g |

### Example 7

### Anhydrous Cosmetic Preparation

100 g of anhydrous composition contains:

| | | |
|---|---|---|
| Compound of formula (XII) | 0.5 | g |
| Beta-carotene | 0.15 | g |
| SiliconeSE-30 ⁽¹⁾ | 10 | g |
| Silicone fluid 345 ⁽²⁾ | 18 | g |
| Silicone fluid 344 ⁽³⁾ | 55.79 | g |
| Borage oil | 10.0 | g |
| Cholesterol | 0.03 | g |
| 2-hydroxy-n-octanoic acid | 0.7 | g |
| Ethanol 95° | 2.0 | g |

⁽¹⁾ Dimethylsilicone polymer MW 50000 D; viscosity 10000 centistokes at 25° C.
⁽²⁾ Cyclic dimethylsiloxane pentamer
⁽³⁾ Dimethylsiloxane tetramer

### Example 8

### Pediatric Preparation

100 g of cream contains:

| | | |
|---|---|---|
| Compound of formula (XV) | 0.3 | g |
| Steareth | 0.1 | g |
| Cetearyl alcol | 0.4 | g |
| Fluid petrolatum | 12.5 | g |
| Waxy petrolatum | 11.0 | g |
| Ceteareth 6-stearyl alcohol | 6.0 | g |
| Superoxide dismutase | 0.008 | g |
| Camellia sinensis extract | 2.9 | g |
| L-methionine | 0.7 | g |
| Demineralized water | q.b. to 100 | g |

### Example 9

### Lenitive Lotion

100 g of lotion contains:

| | | |
|---|---|---|
| Compound of formula (X) | 0.1 | g |
| Ethanol 95° | 30 | g |
| Perfume | 0.3 | g |
| Demineralized water | q.b. to 100 | g |

### Example 10

### Ampoule

A solution of 1 kg of a substance of formula (XI) in 60 litres of bidistilled water is filtered sterilely, dispensed into ampoules, lyophilized under sterile conditions and aseptically sealed. Each ampoule contains 20 mg of active compound.

### Example 11

### Vials for Injection

A solution of 200 g of a substance of formula (XIII) and 5 g of disodium hydrogen phosphate buffered at pH 6.5 is dissolved in 3 litres of bidistilled water containing a little diluted HCl, then filtered sterilely, dispensed in the vials for injection, lyophilized under sterile conditions and aseptically sealed. Each vial for injection contains 10 mg of active compound.

### Example 12

### Suppositories

A mixture of 40 g of a substance of formula (XIV) is melted with 100 g of soybean lecithin and 1400 g cocoa butter, poured into moulds and cooled to obtain suppositories of 40 mg active substance.

### Example 13

### Tablets

A mixture of 2 kg of a substance of formula (V), 4 kg of lactose, 1.2 kg of potato starch, 0.2 kg of talc and 0.1 kg of magnesium stearate is compressed to give tablets each containing 20 mg of active compound.

### Example 14

### Coated Tablets

The tablets prepared similarly to example 13 are coated with sucrose, potato starch, tragacanth gum and dye.

### Example 15

### Capsules

A 4 kg quantity of a substance of formula (VII) is inserted into hard gelatin capsules such that each capsule contains 40 mg of active compound.

1. Compound of formula (I) 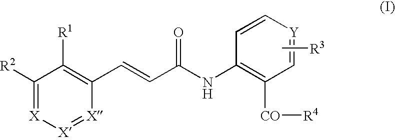
where:
X, X′, X″ and Y each independently represents N,N→O or C—R⁵;
or the X′=X″ group represents a sulfur atom S or an oxygen atom 0, with X remaining as aforedefined, and wherein at least one of X, X′ and X″ is not C—R⁵;
R⁴ represents —OH, O-benzyl, O-phenyl, —CH₂—CH(NHR⁶)—COOR⁸;
R¹, R², R³ and R⁵ each independently represent —H, OH CO—R⁴, —NH₂, —NH—CO—R⁴, —O—COR⁷, -Halogen, —OR⁷, —R⁷;
R⁶ represents H or —COR⁷;
R⁷ and R⁸ each independently represent a linear or branched saturated or unsaturated C₁₋₂₀ alkyl group, optionally substituted: preferably a C₁₋₃ alkyl, even more preferably a methyl group;
or a salt or solvate thereof, excluding the compound N-(o-carboxyphenyl)-β-(3-pyridyl)-acrylamide.

2. Compound as claimed in claim 1 wherein Y is chosen from N and N→O.

3. Compound as claimed in claim 1 wherein at least one of X, X′, X″ is chosen from N and N→O.

4. Compound as claimed in claim 1 wherein X′ and X″ taken together represent a sulfur atom S or an oxygen atom O.

5. Compound as claimed in claim 1, wherein the CO—R⁴ group represents a substituent chosen from: 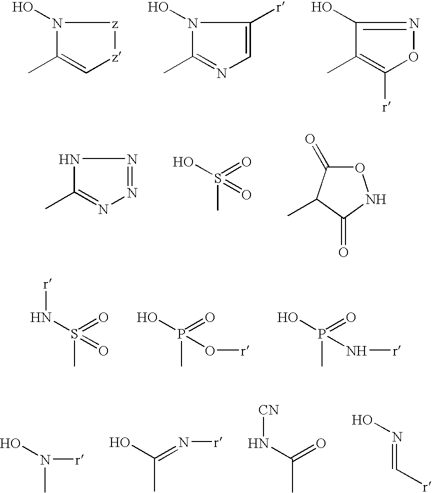
where z and z′ represent N or C-r′; r′ represents H or C₁₋₃ alkyl, preferably H or methyl.

6. Compound as claimed in claim 1, having one of the following structures:

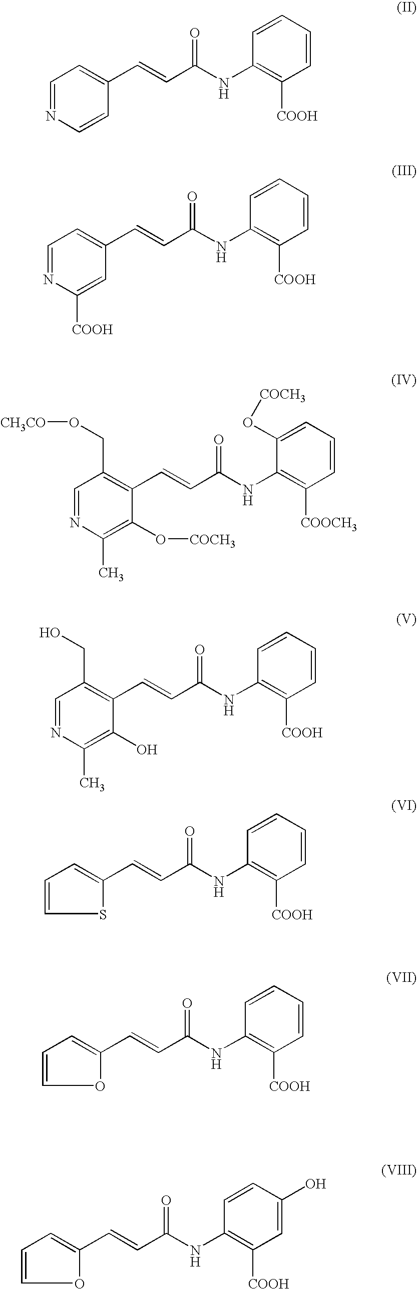

7. Process for preparing a compound of formula (I) 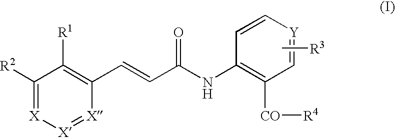
where:
X, X′, X″ and Y each independently represents N,N→O or C—R⁵;
or the X′=X″ group represents a sulfur atom S or an oxygen atom O, with X remaining as aforedefined, and wherein at least one of X, X′ and X″ is not C—R⁵;
R⁴ represents —OH, O-benzyl, O-phenyl, —CH₂—CH(NHR⁶)—COOR⁸
R¹, R², R³ and R⁵ each independently represent-H, OH CO—R⁴, —NH₂, —NH—CO—R⁴, —O—COR⁷, -Halogen, —OR⁷, —R⁷;
R⁶ represents H or —COR⁷;
R⁷ and R⁸ each independently represent a linear or branched saturated or unsaturated C₁₋₂₀ alkyl group, optionally substituted: preferably a C₁₋₃ alkyl, even more preferably a methyl group;
with the proviso that at least one of X, X′, X″ and Y is different from C—R⁵ or if X, X′, X″ and Y are all equal to C—R⁵, then at least one of R¹, R² and R⁵ is different from —H, —OH and —OR⁷;
or a salt or solvate thereof, comprising the condensation of a compound of formula (a) and a compound of formula (b): 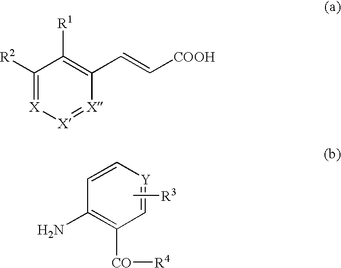
where R¹, R², X, X′, X″, Y, R³, R⁴ have the meanings indicated for formula (I).

8. Process as claimed in claim 7, wherein said condensation is performed in the presence of a coupling reagent which facilitates the coupling of carboxylic moiety with the amine group.

9. Process as claimed in claim 7, wherein said condensation is carried out by reacting compound (b) with compound (a) in activated form.

10. Process as claimed in claim 9, wherein said activated form is the acid halide or anhydride of the compound (a).

11. Use of the compounds of formula (I) as defined in claim 7 for preparing a drug useful in the treatment and/or prevention of dermatological disorders.

12. Use as claimed in claim 11, where the dermatological disorders are of immunoallergic, hyperproliferative or inflammatory type.

13. Use as claimed in claim 12, for the treatment and/or prevention of eczemas, psoriasis, dermatitis, erythemas, fibrosis, lichen ruber planus, pityriasis rosea, autoimmune bullous diseases, urticaria and angioedema.

14. Pharmaceutical composition comprising one or more compounds of formula (I) as defined in claim 7, in therapeutically effective quantities, combined with excipients and dermatologically/physiologically acceptable ingredients.

15. Composition as claimed in claim 14, suitable for administration by topical, oral, intravenous, intraperitoneal, intragastric infusion, enema, infusion via portal vein, inhalation, sublingual, trans-rectal, pulmonary inhalation routes etc.

16. Pharmaceutical composition as claimed in claim 14, comprising the compound of formula (I) in a quantity between 0.01 and 80% by weight of the composition, preferably between 1 and 20% by weight.

17. Pharmaceutical composition as claimed in claim 14, in the form of a lotion, fluid cream, or gel, solution, suspension, emulsion.

18. Cosmetic composition comprising one or more compounds of formula (I) as defined in claim 7, in a cosmetically effective quantity, combined with excipients and ingredients for cosmetic use.

19. Method for cosmetic treatment of the skin, characterised by the topical administration of a cosmetically effective quantity of one or more compounds of formula (I) as defined in claim 7, combined with excipients and ingredients for cosmetic use.
